(19) United States
(12) Patent Application Publication
(10) Pub. No.: US 2023/0084358 A1
(43) Pub. Date: Mar. 16, 2023

(54) FRICTION REDUCTION FOR IMPLANTABLE DEVICES, AND ASSOCIATED SYSTEMS AND METHODS

(71) Applicant: Boston Scientific Scimed, Inc., Maple Grove, MN (US)

(72) Inventors: Padraig J. Savage, Santa Rosa, CA (US); Joshua Mark Inouye, Brooklyn Park, MN (US); Lucas Schneider, Champlin, MN (US); Levi Joel Wolterstorff, Saint Paul, MN (US); Nathan Plag, Minneapolis, MN (US)

(73) Assignee: BOSTON SCIENTIFIC SCIMED, INC., Maple Grove, MN (US)

(21) Appl. No.: 17/942,402

(22) Filed: Sep. 12, 2022

Related U.S. Application Data
(60) Provisional application No. 63/243,458, filed on Sep. 13, 2021.

(51) Int. Cl. A61F 2/24; A61F 2/00

(52) U.S. Cl. CPC A61F 2/2445; A61F 2/2466; A61F 2/0077; A61F 2220/0041; A61F 2250/0008

(57) ABSTRACT

Coating of at least a portion of components of an implantable device to reduce friction during movement therebetween. The surfaces of the components which are coated may be pretreated in a manner contrary to recommended for application of such coating or may simply not be treated as recommend for application of such coating.

### CROSS-REFERENCE TO RELATED APPLICATIONS

This application claims the benefit of priority of U.S. Provisional Application No. 63/243,458, filed Sep. 13, 2021, the entire disclosure of which is hereby incorporated by reference herein for all purposes.

### FIELD

The present disclosure relates generally to the field of implantable medical devices. In particular, the present disclosure relates to medical devices, systems, and methods for cardiac treatment.

### BACKGROUND

Various implantable devices comprise one or more parts which function best if capable of moving relative to one another without friction and/or stiction. However, the parts of some of such implantable devices may also be configured to have an implanted configuration in which the parts should no longer move with respect to one another. As such, it would be desirable to facilitate relative movement of selected parts of an implantable device while also at least somewhat limiting movement of such parts once the implantable device has been implanted so that the implantable device does not shift its implanted configuration and/or back out or otherwise disengage or dislodge from the implant site. For instance, transcatheter mitral valve repair devices (or any cardiovascular device) may be highly complex and the design may have high-friction interfaces. The high friction may cause device failure or difficulty deploying the device. It is desired in many cases to reduce friction as much as possible. However, once the device is implanted, it is generally desirable for the device to remain in the deployed, implanted configuration determined during the implant procedure. In some instances, ease of movement of movable components of an implantable device may not be desirable for an extended period after implantation. Solutions for friction reduction which do not affect the functioning of an implantable device after being implanted would thus be welcome.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an implantable annuloplasty device includes a frame member, and a plurality of movable components movable with respect to the frame member to adjust the shape of the frame member. A friction-reducing coating is applied to at least a portion of a surface of one of the plurality of movable components.

In some embodiments, the plurality of movable components include at least one slider and associated slider screw extending through the slider; and at least a portion of a surface of at least one of the slider or the slider screw is coated with a friction-reducing coating. In some embodiments, the slider screw includes a mounting structure mounted in a window in the frame member, a threaded portion engageable with internal threads within the slider, and a neck between the mounting structure and the threaded portion and extending through an axial opening in the window in the frame member; and the slider is held against axial movement with respect to the frame member and is rotatable with respect to the frame member and the slider to cause axial advancement or retraction of the slider with respect to the frame member; and at least a portion of the slider is coated with a friction-reducing coating. In some embodiments, at least a portion of at least one of the threaded portion, the slider mounting structure, or the slider neck is coated with a friction-reducing coating. In some embodiments, the internal threads of the slider are coated with a friction-reducing coating. In some embodiments, at least one of the sliders is electropolished before application of a friction-reducing coating thereto. In some embodiments, a slider screw associated with the at least one slider is anodized. In some embodiments, the slider screw associated with the at least one slider is coated with a friction-reducing coating.

Additionally or alternatively, the plurality of movable components includes at least one anchor movable with respect to the frame member to secure the frame member to an implant site, and an associated anchor housing mounted to the frame member and through which the at least one anchor extends; and at least a portion of a surface of the anchor housing is coated with a friction-reducing coating. In some embodiments, the anchor housing is smoothened before application of the friction-reducing coating thereto. In some embodiments, the anchor housing is tumbled before application of the friction-reducing coating thereto. In some embodiments, the anchor housing is smoothened before application of the friction-reducing coating thereto.

In some embodiments, the coated component is formed of a metal and the friction-reducing coating is a polytetrafluoroethylene coating.

In some embodiments, the coated surface is an untreated surface which has not been subjected to a pre-treatment process recommended for acceptance of the friction-reducing coating on the surface prior to application of the friction-reducing coating thereto.

In accordance with various principles of the present disclosure, an implantable device and associated deployment system includes an implantable device may include at least one component movable with respect to another component of the implantable device; and a deployment system configured to adjust the position of the at least one movable component, where at least a portion of at least one of the movable component or a component adjacent the movable component is coated with a friction-reducing coating without having been subjected to a pre-treatment process recommended for acceptance of the friction-reducing coating on the surface prior to application of the friction-reducing coating thereto.

In some embodiments, the coated surface is smoothened before application of a friction-reducing coating thereto.

In some embodiments, the implantable device includes a frame member and at least one of a component movable with respect to the frame member to secure the frame member to tissue, or a component movable with respect to the frame member to adjust a configuration of the frame member; and the deployment system includes at least one latch actuator engageable with an associated latch on the at least one movable component, the latch on the movable components not being coated with a friction-reducing coating.

In some embodiments, the coated surface is smoothened before application of a friction-reducing coating thereto.

In accordance with various principles of the present disclosure, a method of reducing friction between components of an implantable device includes applying a friction-reducing coating to at least a portion of at least one component of the implantable device which is movable with respect to another component of the implantable device without pre-treating the portion to which the friction-reducing coating is applied to facilitate acceptance of the friction-reducing coating on the portion.

In some embodiments, the method further includes allowing the efficacy of the friction-reducing coating to be reduced as the movable component is moved.

In some embodiments, the method further includes smoothening the surface of the at least one portion of the at least one component prior to applying the friction-reducing coating thereto.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a schematic view of a human heart valve with an example of an implantable device with an anchoring assembly formed in accordance with various aspects of the present disclosure, the implantable device shown in a compact delivery configuration for delivery to the implant site.

FIG. 2 is a detail view of a portion of an implantable device as in FIG. 1 with a slider thereof in phantom.

FIG. 3 is an exploded view of a slider and slider screw as in FIG. 2.

FIG. 4 is a cross-sectional view of an anchor housing and anchor along line IV-IV of a device as in FIG. 1.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore.

Various medical devices, such as implantable devices, include one or more components which are movable with respect to one another. In accordance with various principles of the present disclosure, movable components of a medical device (which may have complex interfaces) that are susceptible to unwanted friction or stiction (reference being made herein simply to friction for the sake of convenience and without intent to limit) are coated, at least in strategic locations, with a coating. It will be appreciated that reference is generally made herein to application of a friction-reducing coating, although the present disclosure need not be so limited. For instance, the entire surface of a component may be coated, or only a portion or limited surface area of the component, such as at a critical interface, may be coated. A “critical interface” may be considered as an interface (e.g., meeting of surfaces) at which components move relative to one another in order to actively use the device. It will be appreciated that use of a device encompasses implementation, implantation, etc., and the term “actively use” in such context (and various grammatical forms of the term “use”) may be interchanged with terms such as implement, implant, anchor, adjust, cinch, torque, slide, pivot, rotate, etc. without intent to limit. In the context of an implantable device, use of the device herein encompasses use before, during, and after implantation. A critical interface may also encompass interfaces at which, once movement of the device and/or components thereof is no longer desired or needed (even if the device is still in use), it is desirable to inhibit or prevent relative movement between components meeting at such interface. For instance, some implantable devices include movable components which are moved during implantation into body tissue, but which are to remain substantially fixed or immovable relative to one another once the device has been deployed or implanted. Although the implantable device is still considered to be in use, such use may be considered a deployed use or the indicated ultimate use, in contrast with an actively movable use, such as during deployment. It will be appreciated by those of ordinary skill in the art that different implantable devices have different use periods with different requirements with regard to relative movability of components of the implantable device 100, principles of the present disclosure being applicable to at least the period of use during which relative movement of components is desired and/or required, and optionally also to periods after such active period of use during which relative movement of components may not be desirable, as will be clear to those of ordinary skill in the art.

In accordance with various principles of the present disclosure, components of an implantable device may be pre-treated before application of a coating (such as, but not limited to, a friction-reducing or an anti-friction coating), or a coating may be applied to untreated surfaces (surfaces which have not been further modified after formation of the component to accept or otherwise be prepared for application of a coating thereto). Treatment of a surface to facilitate (e.g., enhance) receipt of a coating (e.g., to enhance adherence/adhesion of a coating thereto) generally may include any of a variety of treatments such as anodizing, degreasing, sanding, cleaning with a solvent, sandblasting, applying an initial pretreatment material or coating, etching, etc., or otherwise roughening or texturizing such surface. It will be appreciated that terms such as acceptance, receipt, adherence, adhesion, and the like (in various grammatical forms thereof) used in connection with a coating may be used interchangeably herein without intent to limit. In accordance with some aspects of the present disclosure, as discussed in greater detail below, instead of treating a surface to which a friction-reducing coating is applied to enhance receipt of the coating, the surface is treated in a contrary manner, such as smoothened, polished, tumbled, etc. Once a coating has been applied, the coated component may be cured (e.g., heat treated) to set or stabilize or otherwise to complete the coating process. The coating and heating processes may be repeated as many times as appropriate to give the best performance for the materials used (the component being coated and/or the coating). It will be appreciated that the number of coatings and amount and degree of heating to achieve the desired results in accordance with various principles of the present disclosure may vary and may be determined without undue experimentation by one of ordinary skill in the art.

Various principles of the present disclosure may be applied to an implantable device having movable anchors which may be advanced and/or retracted to secure the device to tissue at a treatment site. It will be appreciated that terms such as secure (and other grammatical forms thereof) may be used interchangeably herein with terms (and other grammatical forms thereof) such as affix, implant, couple, engage, anchor, hold, retain, etc., without intent to limit. Additionally or alternatively, various principles of the present disclosure can be applied to medical devices with movable components allowing the device to be shifted between a collapsed configuration and an expanded configuration, such as to reconfigure the tissue to which the device is secured/in which the device is implanted. It will be appreciated that the term shift (and other grammatical forms thereof) may be used interchangeably herein with such terms as adjust, move, transition, or otherwise (and other grammatical forms thereof) without intent to limit. It will be appreciated that the term collapsed (and other grammatical forms thereof) with respect to the frame may be used interchangeably herein with terms such as retracted, contracted, cinched, and the like (and other grammatical forms thereof), without intent to limit, to refer to a configuration or moving of the frame to a more compact configuration. It will be appreciated that the term compact may be used interchangeably herein with such terms as collapsed or compressed or simply unexpanded (such as with respect to the frame axis) without intent to limit.

In some embodiments, principles of the present disclosure may be applied to complex cardiovascular devices, such as mitral valve repair devices, formed with various components with high-friction interfaces and which have various sliding interfaces where friction reduction is desired. For instance, an annuloplasty device implantable in a cardiac valve annulus to repair and/or to reconfigure the valve annulus by adjustment of one or more components of the implantable device to modify the tissue to which the device is secured would benefit from various principles, aspects, concepts, etc. of the present disclosure.

It will be appreciated that the present disclosure describes various principles, aspects, concepts with respect to an implantable device such as an annuloplasty device, without intent to limit the applicability of the disclosed principles, aspects, and/or concepts to a particular device or device components. In the non-limiting examples disclosed herein, implantable device to which various principles of the present disclosure may be applied includes a frame member. The example of an embodiment of an implantable device may also include a cinch assembly configured to shift the frame member between the collapsed configuration and the expanded configuration. The implantable device may be delivered in a collapsed configuration (e.g., via a delivery catheter) and expanded once delivered to a treatment site (e.g., a heart valve annulus). The implantable device may then be expanded, such as by adjusting a cinch assembly, and implanted. The cinch assembly may be adjusted once the implantable device has been implanted to modify the shape or configuration or otherwise of the tissue to which the device has been implanted. For instance, cinching of one or more components of the cinch assembly to expand or contract one or more portions of the implantable device may modify the configuration of the tissue to which the implantable device is secured. In the case of an angioplasty device, adjustment of one or more components of the cinch assembly adjusts the configuration of the annuloplasty device (e.g., a frame member of the annuloplasty device) and the configuration (e.g., shape, geometry, etc.) of a valve annulus to which the implantable angioplasty device is secured.

An implantable device to which various principles of the present disclosure may be applied may additionally or alternatively include an anchoring assembly configured to anchor the implantable device to the treatment/implant site. The anchoring assembly may include anchors which are adjustable to be implanted into tissue at the treatment site (e.g., cardiac tissue, such as a heart valve annulus, in the case of an annuloplasty device). The anchoring assembly may also include anchor housings via which the anchors may be mounted to the implantable device. The anchors may be movable (e.g., rotatable or translatable) with respect to the implantable device and/or the associated anchor housing (by which the anchor is mounted on the implantable device). Generally, it is desirable to allow more rotation of the anchor proximally into the housing. However, such movement of the anchor generally is accompanied by higher torque. It has been observed that application of coatings in accordance with various principles of the present disclosure generally reduces the torque/rotation of the anchor. In other words, the anchor will become stuck at the same torque which would eventually result in deformation of the anchor, but the amount of rotation imparted to reach such torque is greater with coated housings, allowing the operator to recover from a larger range of rotation he/she may have inadvertently applied to the anchors. Application of coatings in accordance with various principles of the present disclosure has been found to provide a user with a factor of safety the user can recover from, whereas without a coating, any small amount of interaction between the coil and the housing would result in a “locked” anchor and the procedure could not proceed.

Although it is desirable to facilitate movement of the movable components of an implantable device, such as to deploy, implant, adjust, etc., the device, it may also be desirable for such movable components to remain in place once a desired state or configuration of the implantable device has attained.

It will be appreciated that in some instance it may be desirable to eventually inhibit movement of previously movable components (e.g., rotation of the components of a cinch assembly or an anchor with respect to the implantable device) once the desired deployment and implantation of the implantable device (e.g., configuration of the implantable device, such as a frame member thereof, or insertion depth of the anchor into tissue) has been attained. One or more components on which the coating is applied may be treated or left untreated (not treated as recommended, such as by a coating manufacturer, for acceptance of a coating) in a manner which allows the coating to be useful for a limited number of movements or cycles of the coated component relative to another component so that ultimately the coating does not improve movement of the (previously) coated component relative to another component. For instance, if it is generally desirable for the surface of a component to be pre-treated before application of a coating thereto, such component may not be pre-treated in the recommended manner so that the coating efficacy is limited to use during delivery and deployment, but is not as effective once the device has been implanted and left in place for the indicated ultimate use.

It will be appreciated that principles and concepts disclosed herein may be applied to other disparate medical devices, the examples disclosed herein not limiting such further applications of principles of the present disclosure.

Various embodiments of implantable devices coated in accordance with various principles of the present disclosure will now be described with reference to examples illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein.

Turning now to the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element).

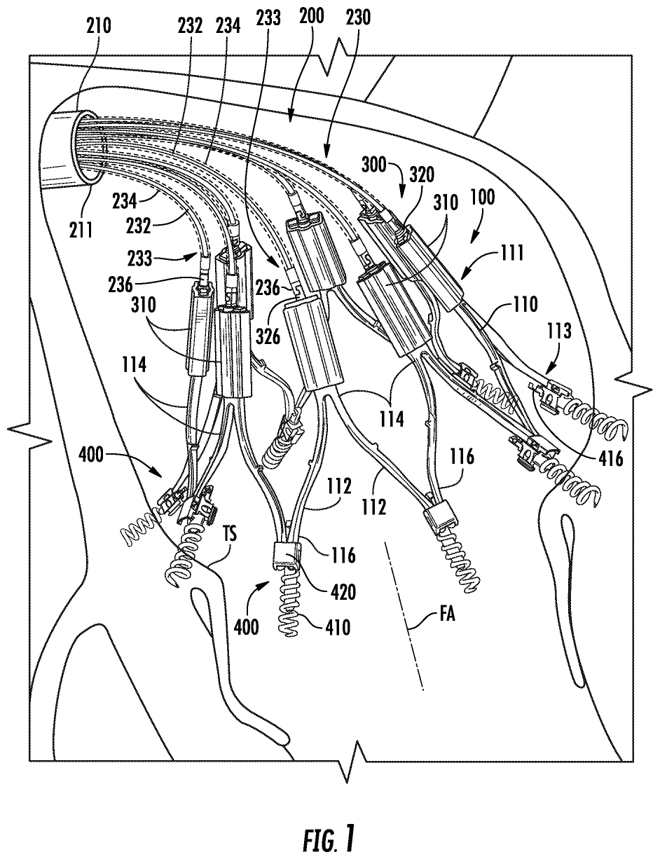

An example of an implantable device 100 which may be formed in accordance with principles of the present disclosure is an implantable annuloplasty device, for custom reshaping of a heart valve (e.g., the mitral valve, or the tricuspid valve), such as illustrated in FIG. 1. It will be appreciated that various principles of the present disclosure are applicable to other forms and types of implantable devices, reference being made to an annuloplasty device as only one example of an implantable device to which principles of the present disclosure may be applied.

The example of an implantable device 100 illustrated in FIG. 1 includes a frame member 110 that may form a generally tubular shape extending about a frame axis FA (indicated for geometrical reference and not for limitation). As used herein, the term “tubular” is to be understood to include circular as well as other rounded or otherwise closed shapes surrounding or enclosing an area. As referenced herein, the frame axis FA is an axis relative to which the frame extends when expanded or contracted. In an embodiment of a frame member 110 which is generally circular, the frame axis FA is a central longitudinal axis of the frame member 110. However, the frame axis FA need not be a central axis. The frame member 110 may be generally symmetrical with respect to the frame axis FA (indicated for geometrical reference and not for limitation), although it need not be symmetrical. As used herein, reference to the “circumference” of the frame member 110 is to be understood as referencing a perimeter or boundary. The frame member 110 may assume various shapes, sizes, dimensions, configurations, etc. during different phases of delivery and deployment such as, without limitation, during pre-delivery, delivery, tissue engagement, anchoring, adjustment (e.g., cinching), etc.

In the illustrated embodiment, the implantable device 100 may be a part of a system configured to be delivered in a minimally invasive manner, such as for example transluminal, percutaneous, endoluminal, etc., delivery (e.g., transfemorally, transeptally, or transapically) to the heart. For instance, the implantable device 100 may be delivered and deployed with a delivery/deployment system 200 which may be configured to deliver the implantable device 100 through tortuous pathways within the body. The delivery/deployment system 200 may include a delivery catheter 210 in which the implantable device 100 may be positioned (e.g., in a compact configuration) until delivery, at which point the implantable device 100 is advanced distally through the distal open end 211 of the delivery catheter 210, and into a delivery (e.g., expanded) configuration such as illustrated in FIG. 1. Accordingly, the implantable device 100 may be delivered in a compact configuration (which may be referenced as a delivery configuration) with a proximal end 111 thereof coupled to/engaged by/carried by the delivery/deployment system 200 and the distal end 113 thereof distal to the delivery/deployment system 200 for engagement with a treatment site TS. The implantable device 100 is expandable (such as in a direction away from the frame axis FA) into an expanded configuration for deployment, placement with respect to the treatment site TS (e.g., cardiac valve annulus), anchoring or securing to the treatment site, etc. The implantable device 100 may expand naturally (e.g., may be self-expandable), for example if the frame is formed of a shape memory or super-elastic material (e.g., Nitinol) that is biased towards an expanded state. Alternatively, or additionally, the implantable device 100 may expand with assistance of an expansion device or mechanism, for example through the use of a force applied within the frame such as using an expandable deployment device (e.g., an inflatable balloon or the like).

The frame member 110 may be configured to change shape, size, dimension, and/or configuration, such as to modify the shape, size, dimension, configuration, etc. of the valve annulus (or other structure) to which it is coupled. The frame member 110 may be formed from a tubular member which is laser cut into the desired shape or configuration. Alternatively, the frame member 110 may be formed from a wire, such as a wire fused together by a laser. The frame member 110 may generally be formed with a plurality of struts 112, which may be components that are formed separately and attached together (optionally permanently, such as by welding or other methods), or different portions of the same, coextensive component. The struts 112 may be arranged with respect to one another in a sinusoidal or zig-zag pattern. The struts 112 may include elongated structural members formed of a metal alloy, a shape memory material, such as an alloy of nickel titanium or other metals, metal alloys, plastics, polymers, composites, other suitable materials, or combinations thereof. In one embodiment, the struts 112 may be formed from the same, monolithic piece of material (e.g., tube stock). Proximal ends of adjacent struts 112 (closer to the delivery/deployment device 200 and further from the tissue to which the implantable device 100 is to be implanted than are the distal ends) may meet at (e.g., be joined to form, or otherwise define) proximal apices 114, and distal ends of adjacent struts 112 (further from the delivery/deployment device 200 and closer to the tissue to which the implantable device 100 is to be implanted than are the proximal ends) may meet at (e.g., be joined to form, or otherwise define) distal apices 116. In some embodiments, the terms “apex,” apices,” and the like may be used interchangeably with terms “crown,” “crowns,” and the like, as used herein and as used in any reference incorporated by reference herein, unless otherwise stated. In one embodiment, an “apex” may include a proximal or distal portion of the frame member. The distal and proximal apices may be spaced sequentially (in an alternating manner) about the circumference of the frame member 110. The frame member 110 may be heat set into a desired shape and/or into a shape for further assembly. For instance, the frame member 110 may be etched (e.g., electrochemically) and/or polished, such as to remove irregular and/or unwanted material and/or to smoothen the surface of the frame member 110. It will be appreciated that alternate configurations of the frame member 110, such as depending on the manner and orientation in which the implantable device 100 is delivered, are within the scope and spirit of the present disclosure.

The example of an implantable device 100 illustrated in the figures includes a cinch assembly 300 configured to engage with the frame member 110 to adjust the configuration of the frame member 110. For instance, a plurality of sliders 310 are carried at the proximal end 111 of the frame member 110, such as along the proximal apices 114 of the frame member 110. It will be appreciated that the term slider may be referenced or otherwise known as collars or sleeves or nuts, with or without the term “cinch”, and such terms may be used interchangeably herein without intent to limit, reference being made generally to sliders 310 for the sake of convenience. It will be appreciated that a slider 310 may be positioned on all or only some of the proximal apices 114. Advancement or withdrawal of a slider 310 with respect to the proximal apex 114 over which the slider 310 is positioned adjusts the relative positions of the struts 112 joined at such apex 114. For instance, distal advancement of the slider 310 towards the distal end 113 of the frame 110 brings together the struts 112 forming the proximal apex 114 over which the slider 310 is mounted to collapse at least a portion of the frame 110 (reduce the overall width of the frame 110), such as towards the collapsed configuration. Likewise, proximal retraction of the slider 310 towards the proximal end 111 of the frame 110 allows the struts 112 to move apart to allow at least a portion of the frame 110 to expand, such as towards the expanded configuration. Each slider 310 preferably is adjustable independently of the other sliders 310. Such adjustment results in adjustment of at least one of the size, shape, configuration, dimension, etc. of the frame member 110 (e.g., retraction, compression, or expansion of the frame upon bringing adjacent struts 112 closer or further apart, respectively) to affect at least one of the size, shape, configuration, dimension, etc. of the treatment site TS (such as to restore or correct the shape of a valve annulus for proper functioning or competency thereof). The implantable device may be delivered into a treatment site TS in a compact configuration. The sliders 310 may be retracted to allow expansion of the frame member 110 upon delivery to the implant site for implantation. Once the device has been implanted, the positions of one or more of the sliders 310 may be adjusted to adjust the configuration of the frame member 110 to affect the configuration of the tissue (e.g., valve annulus) to which the implantable device 100 is anchored.

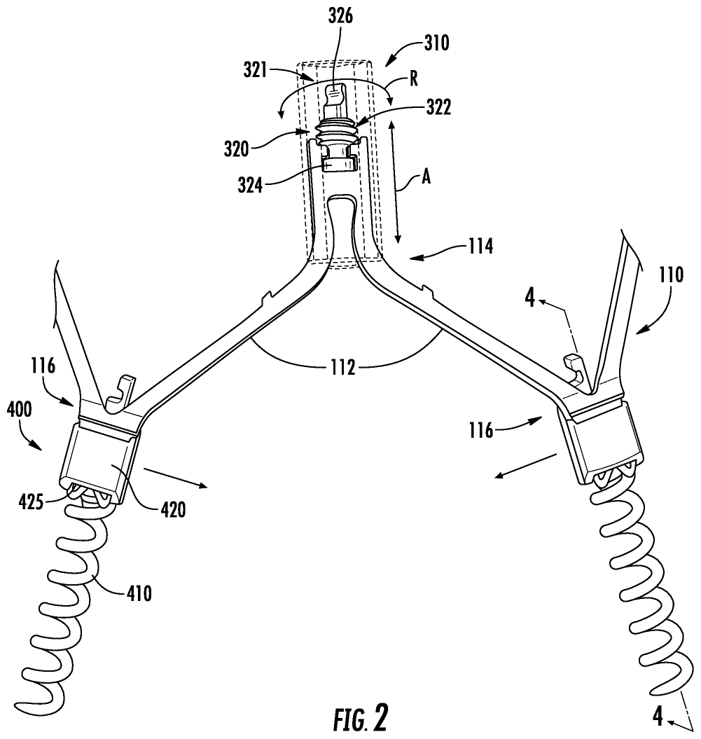

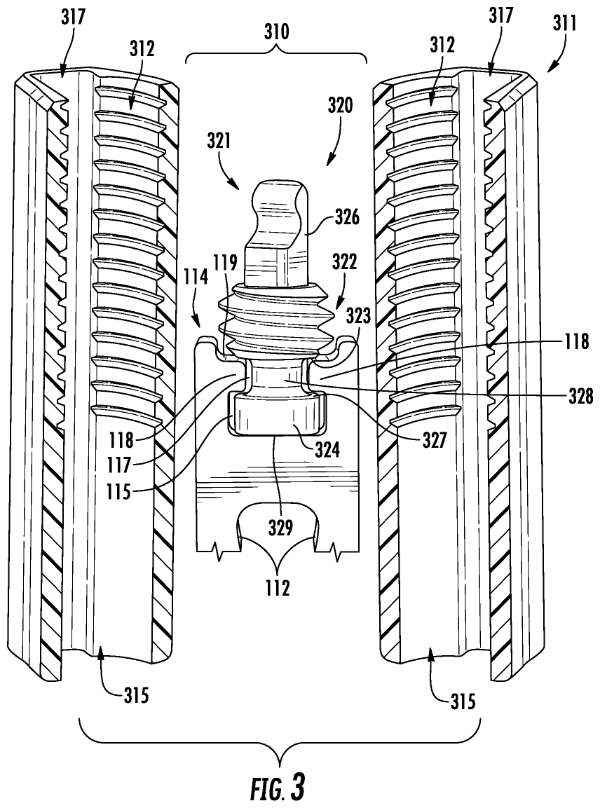

The sliders 310 may be advanced or retracted to adjust the relative positions of struts 112 forming the proximal apex 114 in various manners, such as by engagement with a slider screw 320 extending longitudinally within the slider 310. As illustrated in further detail in FIG. 2 and FIG. 3, the slider screw 320 may include a threaded portion 322 with exterior threads engaging corresponding interior threads 312 within the slider 310. In some embodiments, the slider 310 is held against rotational movement (e.g., about rotational direction R) with respect to the frame member 110, and the slider screw 320 is held against axial movement with respect to the frame member 110 along an axial direction A such that rotation of the slider screw 320 causes axial movement of the sliders 310. For instance, as may be appreciated with reference to the exploded view of FIG. 3, the slider 310 and proximal apex 114 may be shaped to inhibit rotational movement therebetween, such as by having respective noncircular or flat engaging surfaces, such as with respective substantially rectangular cross-sectional shapes. It will be appreciated that the slider 310 illustrated in FIG. 3 is shown as separated halves merely for the sake of illustrating a perspective view of the interior thereof (such as to illustrate internal threads 312), and not otherwise to limit the slider to separate halves. As may be appreciated with reference to FIG. 2 and FIG. 3, the slider screw 320 may include a slider screw mounting structure 324 configured to be coupled with the frame member 110 to hold the slider screw 320 axially along A with respect to the frame member 110. In the example of an embodiment illustrated in FIG. 2 and FIG. 3, the slider screw mounting structure 324 is substantially barbell-shaped and is positioned within a window 115 defined in the proximal apex 114 of the frame member 110 with which the slider screw 320 is mounted. The slider screw 320 may extend through an axial opening 117 of the window 115. The proximal apex 114 of the frame member 110 may include a frame member mounting structure 118 along (e.g., on a side of, such as on either side of) the axial opening 117 to restrain the slider screw mounting structure 324 from exiting the window 115 and thereby inhibiting the slider screw 320 against axial movement (e.g., in a direction A, such as along the frame axis FA) with respect to the proximal apex 114 of the frame member 110.

One or more slider actuators 230 (which may in some aspects be considered a part of the delivery/deployment system 200 and/or part of the cinch assembly 300) may be provided to actuate the sliders 310 as desired, as illustrated in FIG. 1. The actuator may be any known or heretofore known actuator in the art (the structure thereof not being critical to the present disclosure), and may be alternately referenced herein as a driver or controller (with or without the term “mechanism”), or a driver mechanism or control mechanism without intent to limit. It will be appreciated that the term actuate (including other grammatical forms thereof) may be used interchangeably herein with such terms (and other grammatical forms thereof) as control, maneuver, manipulate, move, operate, drive, shift, transition, advance, retract, rotate, translate, etc., without intent to limit. One or more of the slider actuators 230 may include a flexible elongate member 232 (e.g., extending through a lumen defined in a tubular flexible elongate member 224, shown in phantom and described in further detail below) with a slider actuator latch 236 at a distal end 233 of the flexible elongate member 232. The slider actuator latch 236 is configured to engage a slider screw latch 326 (shown in greater detail in FIG. 2 and FIG. 3) on a proximal end 321 of the slider screw 320. Rotation of the slider actuator 230 rotates the slider screw 320 to cause translation of the associated slider 310 as described above. The slider actuator 230 may be actuated in any manner known to those of ordinary skill in the art, such as via a control knob at a proximal end thereof, such as known in the art and not illustrated for the sake of simplicity, the configuration of which not being critical to the principles of the present disclosure. A slider latch cover 224, such as in the form of a tubular flexible elongate member 224 (e.g., a hollow shaft or tube or hypotube) may extend over the slider actuator latch 236 and the slider screw latch 326 to hold the slider actuator latch 236 with respect to the slider screw latch 326 to transmit torque thereto. The slider latch cover 224 may be retracted to allow disengagement/decoupling of the slider actuator latch 236 and the slider screw latch 326 once the desired position of the slider 310 has been attained and/or the implantable device 100 is implanted as desired/medically indicated.

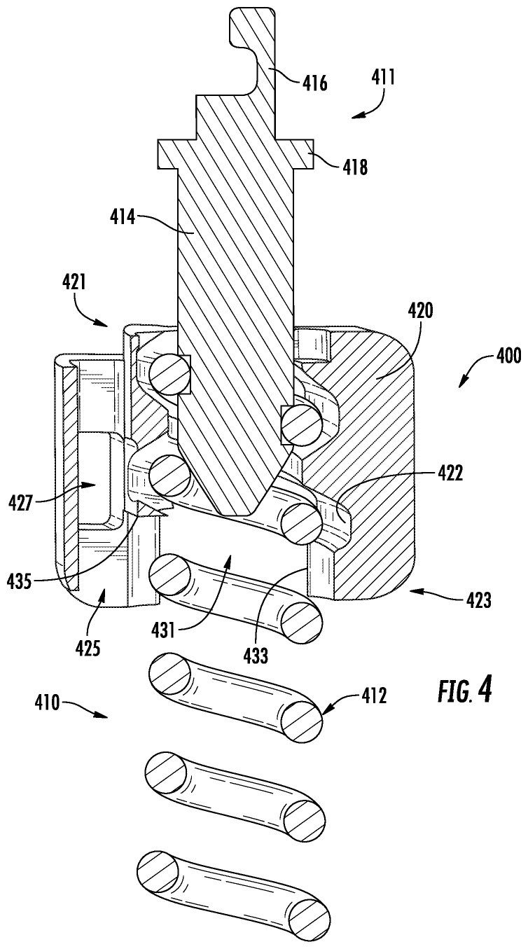

Once the implantable device 100 is in a deployment configuration, the implantable device 100 may be anchored with respect to the implant site/treatment site TS (in the embodiment illustrated in FIG. 1, a heart valve annulus) with one or more anchor assemblies 400, examples of which are illustrated in further detail in FIG. 4. In the illustrated embodiment, the anchor assemblies 400 are provided at a distal end of the implantable device 100, such as at a distal end 113 of the frame member 110, to anchor the implantable device 100 with respect to the treatment site TS. In accordance with various principles of the present disclosure, at least one of the anchor assemblies 400 includes at least one anchor 410, configured to secure the implantable device 100 to the treatment site TS. In some embodiments, the anchors 410 are movable with respect to the frame member 110 of the implantable device 100. The anchors 410 may extend distally from the frame member 110 and may have sharpened distal tips to penetrate and to facilitate entry and advancement into tissue at the treatment site TS. In some embodiments, the anchor assembly 400 includes an anchor housing 420 via which the anchors 410 are mounted or coupled to the frame member 110. The anchor housings 420 may be coupled to the frame member 110 such as by receiving a portion (e.g., a distal apex 116) of the frame member 110 through an opening or frame slot 425 in the anchor housing 420. It will be appreciated that the term frame slot is used for the sake of convenience and may be used interchangeably herein with terms such as frame channel, frame sleeve, or the like, without intent to limit. The anchor housing 420 may be fixed against relative movement with respect to the frame member 110 in accordance with any manner known in the art, such as a mechanical engagement such as a friction fit or interference fit (e.g., engagement of lateral or circumferentially extending tabs on the distal apex 116 of the frame member 110 within windows 427, shown in FIG. 4, in the anchor housing 420/frame slot 425) or by welding or the like, such as between the distal apex 116 of the frame member 110 and the anchor housing 420 (e.g., the frame slot 425), the particular manner of retention not being critical to the principles of the present disclosure.

The anchors 410 may include an anchor shaft such as in the form of helical coil with a plurality of turns 412 and an anchor head 414. It will be appreciated that the anchor shaft may be in any of a variety of other configurations instead of a substantially helical coil as illustrated, such as a substantially elongated element with external threads thereon, reference being made herein to “turns” (of a coil or threads) for the sake of convenience and without intent to limit. The anchor 410 advances or retracts through an anchor bore 431 defined through (e.g., axially through) the anchor housing 420. The anchor 410 may be guided within the anchor bore 431 of the anchor housing 420 by mating of the turns 412 of the anchor shaft with internal threads or grooves 422 on the inner wall in the anchor bore 431, as illustrated in FIG. 4. The anchor bore 431 may include an unthreaded distal section 433 (closer to the distal end 423 of the anchor housing 420) in which the proximal turns 412 of the anchor 410 may be positioned when the anchor 410 is substantially fully deployed to allow for “free spin” of the anchor (drawing tissue proximally towards the anchor housing 420 without advancing the helical anchor 410 distally with respect to the anchor housing 420). More particularly, in some embodiments, the helical anchor 410 may be distally advanced into the tissue until the proximal-most turn of the helical coil 410 is positioned within the unthreaded distal section 433 in the anchor bore 431 of the anchor housing 420. Once the turn 412 is no longer engaged with the threads 422 in the anchor housing 420, further rotation of the helical anchor 410 does not result in further distal advancement of the helical anchor 410, but may result in tissue being drawn proximally towards the anchor housing 420 to improve affixation of the anchor assembly 400 to the tissue. Undesired distal advancement of the anchors 410 through (and beyond or out of) the anchor housing 420 may be prevented by abutment of an anchor head shoulder 418 (such term may be used interchangeably herein with flange or projection or the like without intent to limit) on the anchor head 414 with a proximal end 421 of the anchor housing 420, as may be appreciated with reference to FIG. 4. An internal shoulder 435 may be provided at a proximal end of the unthreaded distal section 433 of the anchor bore 431 to prevent or inhibit unintended or undesired proximal withdrawal of the anchor 410 from the anchor housing 420. Various additional features of anchor assemblies may be appreciated with reference to the following patents and patent applications, each of which is incorporated herein by reference in its entirety for all purposes: U.S. Pat. No. 10,548,731 to Lashinski et al., titled Implantable Device and Delivery System for Reshaping a Heart Valve Annulus, and in U.S. Patent Application Publication 2021/0068955, published on Mar. 11, 2021, and titled Spring Loaded Self Locking Reversible Anchor, and U.S. provisional patent application ______ [ATTORNEY DOCKET 8150.0758Z], filed Dec. 17, 2020, and titled Anchoring Devices, Assemblies, And Methods For Implantable Devices.

The anchors 410 may be actuated to advance or retract in any of a variety of manners, such as with an anchor driver assembly which may in some aspects be considered a part of the delivery/deployment system 200. It will be appreciated that the anchor driver assembly may include anchor actuators 230 similar to the slider actuators 230 in form and/or function and reference is made to the description of the slider actuators 230 as generally applicable to the anchor actuators 230, and may in some aspects be considered a part of the delivery/deployment system 200). For the sake of simplifying and streamlining FIG. 1, the components of an anchor actuator are not illustrated, those of ordinary skill being readily able to appreciate the structure, configuration, arrangement, function, etc., of components thereof with reference to the slider actuator 220 as illustrated in FIG. 1.

Once the implantable device 100 has been anchored to tissue (e.g., a treatment site TS such as a heart valve), the anchor actuators may be decoupled from the anchors 410. Optionally, the configuration of the anchored implantable device 100 may be adjusted to modify the tissue to which the implantable device 100 is anchored. For instance, one or more sliders 310 may be adjusted relative to a frame member 100 of the implantable device 100 to adjust the configuration of the frame member 110 of the implantable device 100 and thereby to modify the configuration of the tissue to which the implantable device 100 is anchored. Once the desired configuration (of the implantable device 100 and the tissue to which the implantable device 100 is anchored) has been achieved, the slider actuators 230 may be decoupled from the sliders 310, such as by proximally withdrawing the slider latch cover 224 to allow disengagement/decoupling of the slider actuator latch 236 and the slider screw latch 326.

It will be appreciated that it is generally desirable for the sliders 310 and slider screws 320 to move with relative ease with respect to one another (so that such components do not stick, and the torque which must be applied to an actuator to actuate the slider screws 320 and sliders 310 remains at an acceptable level for a medical professional, such as 0.25 to 5 in-o). It will further be appreciated that although the sliders 310 and slider screws 320 generally are not subjected to external forces which may affect the relative positions of the sliders 310 and slider screws 320 and/or to affect the positions of the sliders 310 relative to the frame member 110 once the implantable device 100 has been implanted and left in place, it is desirable for any movement of the sliders 310 and slider screws 320 to be minimized after implantation of the implantable device 100.

Similarly, it will be appreciated that it is generally desirable for anchors 410, such as those moved with respect to an implantable device 100, will move with relative ease during implantation to secure the implantable device 100 to tissue. However, once the implantable device 100 has been secured to tissue, it is generally desirable for any movable anchors 410 used to secure the implantable device 100 to be generally held in place and not to move further or dislodge.

In accordance with various principles of the present disclosure, one or more contact areas between one or more components of an implantable device 100 is coated to improve relative movement therebetween. For instance, a friction-reducing coating may be applied to at least a portion of one or more surfaces of a component, such as a movable components or component adjacent to (and generally contacting) a movable component of an implantable device 100. For instance, in the embodiment of an implantable device 100 illustrated in FIGS. 1-4, at least one or more of the sliders 310, slider screws 320, anchors 410, or anchor housings 420, and optionally also an area of the frame member 110 adjacent one or more such components is coated to improve relative movement therebetween. A coating, such as a friction-reducing coating, applied in accordance with various principles of the present disclosure may be selected from any of a variety of coatings (e.g., lubricious, biocompatible, etc.), such as polytetrafluorethylene (PTFE), and, more particularly pure low molecular weight PTFE, optionally dispersed in a solvent. The coating/coated component may be treated after application of the coating to the component. In some embodiments, the coating is heat treated, such as fused at high temperatures, which has been found to greatly reduce the friction at critical interfaces of the implantable device 100 (e.g., at least an approximately 20% reduction, and even as high as at least an approximately 60% reduction in friction). In one example, Tiolon® X-20 pure low molecular weight PTFE dry film coating (sold by Tiodize® Company, Inc.) is used. Other options for coatings include, without limitation, Xylan®, PEEK®, KYNAR®, HALAR®, TEFLON® (with risk analysis assessment), or Nylon coatings.

It will be appreciated that in some embodiments, the coating is not applied to the slider screw latch 132 or to the anchor latch 416 to facilitate secure engagement therewith with an associated actuator latch of an associated respective slider actuator or anchor actuator.

In accordance with various principles of the present disclosure, a coating is applied to at least a portion of a surface of one or both of at least one slider 310 and/or an associated slider screw 320 (positioned to actuate the at least one slider 310) and/or adjacent portions of the frame member 110 formed in accordance with various principles of the present disclosure. For instance, a friction-reducing coating may be applied to at least a portion of one or more surfaces of the slider 310 and/or slider screw 320, such as surfaces which are generally subjected to high load conditions with respect to the frame member 110/proximal apex 114 of the frame member 110 as the cinch assembly 300 is actuated (with the slider screw 320 being moved with respect to the slider 310 and frame member 110). For instance, in the example of an embodiment illustrated in FIG. 2 and FIG. 3, during actuation of the slider screw 320, high load conditions may be observed between the slider screw 320 and the proximal apex 114 in which the slider screw 320 is mounted, and, even more particularly, the window 115 of the proximal apex 114 in which the slider screw 320 is mounted, particularly if there is a tight fit upon mounting the slider screw 320 on the proximal apex 114. In some embodiments, a friction-reducing coating is applied to at least a portion of a proximal surface 327 of the slider screw mounting structure 324, and, optionally, to at least a portion of the adjacent surface of the window 115. Additionally or alternatively, a friction-reducing coating may be applied to at least a portion of a distal surface 329 of the slider screw mounting structure 324 and, optionally, to at least a portion of the adjacent surface of the window 115. Additionally or alternatively, a friction-reducing coating may be applied to a distal surface 323 of the threaded portion 322 of the slider screw 310 which may contact a proximal surface 119 of the frame member mounting structure 118 and/or to at least a portion of the proximal surface 117 of the frame member mounting structure 118. In some embodiments, a friction-reducing coating is applied along at least a portion of the threaded portion 322 of the slider screw 320. Additionally or alternatively, a friction-reducing coating may be applied to at least a portion of the neck 328 of the slider screw 320 between the slider screw threaded portion 322 and the slider screw mounting structure 324, and/or to the surface of the axial opening 117 through which the neck 328 extends. It will be appreciated that other surfaces of the slider screw 320 may be uncoated, or remain uncoated. It has been found that lower torques required to rotate the slider screw 320 with respect to the slider 310 reduce windup of the slider actuators 230 to actuate the slider screws 320 to actuate the sliders 310, and a coating (such as a friction-reducing coating such as a PTFE coating) significantly increases the smoothness of rotation and reduces the system jitter. It will be appreciated that other surfaces (including just portions thereof) of components of the cinch assembly 300 and associated components of the implantable device 100 and delivery/deployment system 200 may be coated to similar advantage.

Additionally or alternatively, in accordance with various principles of the present disclosure, a coating may be applied to at least a portion of a surface of an anchor assembly 400 formed in accordance with various principles of the present disclosure. For instance, a coating may be applied to an anchor housing 420 through which an anchor 410 rotates or translates to be advanced into tissue or to be retracted therefrom, such as to anchor an implantable device 100 to tissue. In some embodiments of anchor assemblies 400, there is a very small clearance between the anchor 410 and the anchor housing 420, resulting a relatively tight fit which may interfere with use of the anchor assembly 400, such as for adjusting the anchor assembly 400 (during preparation and/or intraprocedurally) and/or for anchoring a device via the anchor assembly 400, and potentially resulting in the anchor 410 binding in the anchor housing 420. Generally, anchors 410 and associated anchor housings 420 of implantable devices 100 (such as annuloplasty devices) are formed of a metal such as stainless steel (e.g., medical grade stainless steel, 316 stainless steel, cobalt chromium alloys, elgiloy, nitinol, titanium alloys, etc.) or titanium. Relative movement of components formed of the same or similar metal with respect to each other may cause rubbing resulting in friction/stiction. A friction-reducing coating on the anchor housing 420 and/or the associated anchor 410 facilitates adjustment of the anchor 410. It has been found that application of a friction-reducing coating in accordance with various principles of the present disclosure has advantageously reduced anchor binding, such as in anchor housings (e.g., if the anchor is proximally backed out intra-procedurally too far from the tissue, such as during repositioning or inadvertent manipulation of a given anchor, and/or during device preparation for implantation). For instance, in some embodiments, when the anchor 410 is fully seated proximally in the anchor housing 420, there is generally no clearance, and adding a coating provides a small layer between the two stainless steel surfaces that would otherwise bind together.

It will be appreciated that anchors 410 formed in accordance with various principles of the present disclosure need not be coated or electropolished or otherwise treated. In general, reduced friction with respect to anchors 410 of an implantable device 100 may not be desirable as reduced friction may adversely impact the secure engagement of the anchors 410 within the tissue in which the anchors 410 are implanted. The turns 412 of the anchors 410 may be stainless steel as drawn (e.g., as a helical wire) over a mandrel, using pulse processing to sharpen the tissue-penetrating tip of the coil, and welded to the anchor head 414. As such, the surface chemistry or roughness of the anchors 410 need not be changed or modified to impact friction with respect to the anchor housing 420.

As described above, although it may generally be desirable to reduce friction between movable components of an implantable device during delivery, deployment, and adjustment thereof, a degree of friction may be desirable once the device is implanted for extended use with the desired position and/or configuration for the ultimate use having been attained. In accordance with various principles of the present disclosure, a coating may be applied to at least a portion of a surface of an implantable device 100 which has not been pretreated to receive such coating. In particular, it is generally advisable to apply coatings such as friction-reducing coatings (e.g., PTFE coatings) to a pre-treated surface, such as a roughened or texturized surface (e.g., an anodized surface in the case of a metal component such as a titanium or titanium alloy component) which facilitates adhering of the coating thereto and may also contribute to longer use of the coating (the coating lasts longer for its intended friction-reducing use). However, in accordance with various principles of the present disclosure, in contrast with general practice, a coating may be applied to a surface not pre-treated or otherwise configured per coating-manufacturer instructions or recommendations to achieve a unique effect desirable for the particular needs for an implantable device. More particularly, in accordance with various principles of the present disclosure, at least some surface areas of movable components are not pre-treated for coating purposes, as may be otherwise recommended for coating a surface. In some instances, a surface of an implantable device 100 to which a friction-reducing coating is applied may be polished or otherwise smoothened in contrast with being roughened as may be typical for application of a coating thereto. It will be appreciated that various coated devices and/or components thereof have a use period known to those of ordinary skill in the art in which such devices/components are used. For instance, in some instances, such as with implantable devices (such as described herein), a device and/or components thereof are moved during a preliminary stage of use (e.g., delivery, deployment, and implantation of an implantable device), but such movement is unnecessary, and even contraindicated in some cases, after such preliminary stage of use. For example, some devices are intended to move or last for a determined use period, such as for a selected number of cycles of movement (e.g., extension/retraction, number of rotations, etc.), during which performance without interference by friction is desirable, and after which such movement is no longer necessary and may even be undesirable. For instance, it may be desirable for implantable devices formed in accordance with various principles of the present disclosure to perform up to five (5) cycles of movement with up to twenty (20) rotations for each half-cycle of anchors and screws. In the case of implantable devices, increased friction may be desirable to counteract various anatomical movements (such as palpatory forces of the heart which may affect implantable cardiac devices) which may affect the long-lasting securement of the implantable device to anatomical structure. It has been found that application of a friction-reducing coating to a surface not pre-treated as generally recommended for optimal receipt of the coating allows the coating to achieve the desired friction reduction during the intended use period during which movability of components is desired, while having a reduced friction-reducing effect towards the end of such use period, with potentially further reduced friction-reducing effect once movement of such coated component generally is no longer desired. For instance, a coating applied to a surface which has not been pre-treated or is not otherwise configured for optimal receipt of the coating may wear away to a desired extent and/or in a desired manner to reduce a friction-reducing result at the end of the active use period of the coated component during which movability of one or more components thereof is desired is complete. After the desired duration or number of use cycles, efficacy of the friction-reducing coating may be reduced. As such, movement of such movable component relative to an adjacent component is reduced after the implantable device 100 has been implanted and the delivery/deployment system 200 has been withdrawn.

In accordance with various principles of the present disclosure, the surface of a slider 310 is not treated as recommended or is pre-treated in a manner contrary to as recommended for application of a coating thereto. For instance, the surface of the slider 310 may allow a friction-reducing coating to be applied thereto, but may not allow such friction-reducing coating to last past implantation of the implantable device 100 so that the slider 310 does not readily move out of its selected final positions (i.e., the positions determined for implantation, such as positions achieving the desired configuration of an implantable device 100 on which the slider 310 is mounted and which is affected by the positions of the slider 310 relative thereto). In some embodiments, a slider screw 320 formed in accordance with various principles of the present disclosure is formed of a titanium base metal and is anodized with AMS2488 Type II anodization, followed by application of Tiolon® X20 which may be applied and fused at a high temperature twice. A corresponding slider 310 formed of a stainless steel base material is not anodized prior to application and fusing of the Tiolon® X20 coating (optionally twice). In such embodiment, a torque reduction of approximately 60% compared to the torque required to rotate uncoated slider screws with respect to associated sliders was achieved. It will be appreciated that in some embodiments, the anodized coated slider screw 320 may be used with an anodized coated slider 310, or an unanodized coated slider screw 320 may be used with an unanodized coated slider screw 320. In some embodiments, the surface of one or more sliders 310 formed in accordance with various principles of the present disclosure may be smoothened, such as electropolished, prior to coating. Such manner of polishing may impact the entire component, including the inner surfaces thereof such as the slider interior threads 312. In some embodiments, the slider 310 may be electropolished, but not at the proximal end 311 thereof (see, e.g., FIG. 3) so that the coating lasts on the proximal end 311 of the slider 310 in case such end of the slider encounters patient anatomy during use after implantation. In some embodiments, it may be considered desirable and/or important to coat the slider interior threads 312 to facilitate movement of the slider screw 320 therethrough. A mandrel may be used to hold the slider 310 during the coating process in such a manner that the coating reaches at least the interior threads 312 of the slider 310. For instance, longitudinal slots 317 defined in the passage 315 within the slider 310 (see, e.g., FIG. 3) for accommodating the proximal apex 114 of the frame member 110 may be provided along either or both sides of the interior threads 312 within the slider 310, and a mandrel may be positioned within such slots 317 to leave the interior threads 312 unimpeded for application of coating thereto. As such, a friction-reducing coating is applied to facilitate movement, at least during deployment and adjustment, between the sliders 310 and the slider screws 320.

In some embodiments, a slider screw 320 formed in accordance with various principles of the present disclosure may be anodized to accept application of a coating thereto. At least the portion of the slider screw 320 which extends through the slider 310 may be coated. In some embodiments, the slider screw 320 is held by the slider screw latch 326 thereof during the coating process, as coating of the slider screw latch 326 generally is not critical to achieve the desired friction reduction for use of the cinch assembly 300 (and may, in fact, be considered undesirable for achieving a firm engagement with a slider actuator latch 236 of a slider actuator 230, such as illustrated generally in FIG. 1).

In some embodiments, an anchor housing 420 formed in accordance with various principles of the present disclosure is not treated as recommended or is pre-treated in a manner contrary to as recommended for application of a coating thereto. In some embodiments, an anchor housing 420 formed in accordance with various principles of the present disclosure is formed from stainless steel (such as 316 Stainless Steel, more particularly, meeting the requirements of ASTM F138/F139) or titanium (e.g., ELI Grade 23). It is desirable to increase the amount of torque (such as in a counter-clockwise direction) that results in a “stuck” anchor 410 (binding of the anchor 410 within an anchor housing 420), such as to increase the degree to which an anchors 410 may be rotated within an associated anchor housing 420. In accordance with various principles of the present disclosure, an anchor housing 420 is coated with a friction-reducing coating to facilitate advancement and retraction of an anchor 410 extending therethrough. Typically, it is recommended to pre-treat a stainless steel component (such as by bead blasting, e.g., with fine ceramic particulate) to improve adherence of the coating thereto. However, bead blasting of implantable components such as anchor housings 420 may introduce other issues. For instance, micro-bead or bead blasting may increase the surface roughness to a point which may contribute to corrosion affects, as well as increased friction. Electropolishing does not always sufficiently remove sharp edges, and the anchor housing may thus be left with a traumatic edge. In accordance with various principles of the present disclosure, an anchor housing 420 formed in accordance with various principles of the present disclosure may be smoothened, such as tumbled, such as to clean up or dull sharp ends (e.g., rounding or forming a billet on a shoulder of the anchor housing 420). Coating of a smoothened (e.g., tumbled) anchor housing 420 in accordance with various principles of the present disclosure generally is contrary to general coating practices, but has been found to provide the desired reduced-friction effect for the desired duration or number of use cycles or use period.

Advantageously, coating of an anchor housing 420 may be achieved such as by a dip process or a spray process or a sputter process. For example, the anchor housing 420 may be racked (e.g., mounted on a rack extending through an opening in the anchor housing 420), dipped in a bath of coating material, and put in an oven to cure. The opening by which the anchor housing 420 is held or racked may be selected to minimally interfere with the coating process. For instance, racking may be through the frame slot 425 and windows 427. Because the frame slot 425 and windows 427 may be configured to receive a portion of the frame member 110 (e.g., a proximal apex 114 of the frame member 110 and optionally tabs thereof in the windows 427) to mount the anchor housing 420 on the frame member 110, such opening preferably ensures tensile strength between the frame member 110 and the anchor housing 420 and coating thereof thus generally is undesirable.

Anodization of an anchor housing 420 formed from titanium in accordance with various principles of the present disclosure has been shown to provide an approximately 90% benefit over non-anodized (e.g., clean or contaminated) stainless steel. It will be appreciated that such measurements are in conjunction with uncoated anchors 410 (coating of an anchor 410 may generally be deemed unnecessary). However, an anodized anchor housing 420 may require larger clockwise torque to free an anchor 410 extending therethrough compared with an anchor housing 420 coated with an anti-friction coating such as Tiolon® X20. In particular, a stainless steel anchor housing 420 formed in accordance with various principles of the present disclosure and coated with an anti-friction coating such as Tiolon® X20 has been observed to provide an approximately 135% increase in counterclockwise torque that results in a stuck anchor 410 extending therethrough. An anodized titanium anchor housing 420 formed in accordance with various principles of the present disclosure and coated with an anti-friction coating such as Tiolon® X20 has also been observed to provide an approximately 135% increase in counterclockwise torque that results in a stuck anchor 410 extending therethrough Such improvements are with respect to an uncoated stainless steel (as machined) as the baseline. In sum, machined titanium anchor housings 420 that have been anodized provide a 90% improvement, whereas machined Stainless steel anchor housings 420 coated with Tiolon® X20, as well as machined titanium anchor housings 420 that were anodized and coated with Tiolon® X20 provide a 135% improvement. It has been observed that stainless steel and titanium anchor housings 420 with Tiolon® X20 coatings have exhibited approximately two full counterclockwise rotations of the anchor actuators 230 to result in binding of an anchors 410 within an associated anchor housing 420, which is approximately 2.5 times greater than the rotations resulting in binding of an anchors 410 within a non-coated anchor housing 420. Although anodization, alone, of a titanium anchor housing 420 allows for greater clockwise rotation of the anchor actuator 230 before an associated anchors 410 binds therein, larger clockwise rotations are required with such embodiment to free the anchors 410 as compared with a coated anchor housing 420 (e.g., an anodized titanium anchor housing 420 coated with Tiolon® X20). It has been observed that stainless steel anchor housings 420 coated with an anti-friction coating such as Tiolon® X20 may have a higher repeatability than titanium anodized anchor housings 420 coated with an anti-friction coating such as Tiolon® X20.

Further details of examples of implantable devices, frame members, sliders, anchor housings, and anchors, and further components and features thereof, and associated delivery devices and methods of use may be appreciated with reference to the following patents and patent applications, each of which is incorporated herein by reference in its entirety for all purposes: U.S. Pat. No. 9,180,005 (Docket No. 8150.0563), issued Nov. 10, 2015, and titled “ADJUSTABLE ENDOLUMINAL MITRAL VALVE RING”; U.S. Pat. No. 10,335,275 (Docket No. 8150.0570), issued Jul. 2, 2019, and titled “METHODS FOR DELIVERY OF HEART VALVE DEVICES USING INTRAVASCULAR ULTRASOUND IMAGING”; U.S. Pat. No. 9,848,983 (Docket No. 8150.0568), issued Dec. 26, 2017, and titled “VALVE REPLACEMENT USING ROTATIONAL ANCHORS”; U.S. Pat. No. 10,555,813 (Docket No. 8150.0571), issued Feb. 11, 2020, and titled “IMPLANTABLE DEVICE AND DELIVERY SYSTEM FOR RESHAPING A HEART VALVE ANNUL US”; U.S. Pat. No. 10,548,731 (Docket No. 8150.0572), issued Feb. 4, 2020, and titled “IMPLANTABLE DEVICE AND DELIVERY SYSTEM FOR RESHAPING A HEART VALVE ANNULUS”; U.S. Pat. No. 9,192,471 (Docket No. 8150.0564), issued Nov. 24, 2015, and titled “DEVICE FOR TRANSLUMENAL RESHAPING OFA MITRAL VALVE ANNUL US”; U.S. Patent Application Publication No. 2010/0249920 (Docket No. 8150.0564X), published Sep. 30, 2010, and titled “DEVICE FOR TRANSLUMENAL RESHAPING OF A MITRAL VALVE ANNULUS”; U.S. Pat. No. 9,795,480 (Docket No. 8150.0565D), issued Oct. 24, 2017, and titled “RECONFIGURING TISSUE FEATURES OFA HEART ANNULUS”; U.S. Pat. No. 9,610,156 (Docket No. 8150.0566), issued Apr. 4, 2017, and titled “MITRAL VALVE INVERSION PROSTHESES”; and/or U.S. Pat. No. 10,321,999 (Docket No. 8150.0569), issued Jun. 18, 2019, and titled “SYSTEMS AND METHODS FOR RESHAPING A HEART VALVE”. Thus, the description of particular features and functionalities herein is not meant to exclude other features and functionalities, such as those described in the references incorporated herein by reference or others within the scope of the development.

It is to be understood by one of ordinary skill in the art that the present discussion is a description of broad principles and aspects of coatings applied to illustrative examples of embodiments only, and is not intended as limiting the broader coating principles and aspects of the present disclosure to the illustrated and described embodiments. These examples are not the only way to implement these principles but are merely examples, not intended as limiting the broader aspects of the present disclosure. Thus, references to elements or structures or features in the drawings must be appreciated as references to examples of embodiments to which the principles and aspects of the present disclosure may be applied, and should not be understood as limiting the disclosure to the specific elements, structures, or features illustrated. Other examples of manners of implementing the disclosed principles and aspects will occur to a person of ordinary skill in the art upon reading this disclosure.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied with other elements, materials, and components, without departing from the concept, spirit, or scope, or characteristics thereof. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable annuloplasty device comprising:
a frame member; and
a plurality of movable components movable with respect to the frame member to adjust the shape of the frame member;
wherein a friction-reducing coating is applied to at least a portion of a surface of one of the plurality of movable components.

2. The implantable annuloplasty device of claim 1, wherein:
the plurality of movable components include at least one slider and associated slider screw extending through the slider; and
at least a portion of a surface of at least one of the slider or the slider screw is coated with a friction-reducing coating.

3. The implantable annuloplasty device of claim 2, wherein:
the slider screw includes a mounting structure mounted in a window in the frame member, a threaded portion engageable with internal threads within the slider, and a neck between the mounting structure and the threaded portion and extending through an axial opening in the window in the frame member;
the slider is held against axial movement with respect to the frame member and is rotatable with respect to the frame member and the slider to cause axial advancement or retraction of the slider with respect to the frame member; and
at least a portion of one of the slider threaded portion, the slider mounting structure, or the slider neck is coated with a friction-reducing coating.

4. The implantable annuloplasty device of claim 3, wherein at least a portion of the slider is coated with a friction-reducing coating.

5. The implantable annuloplasty device of claim 4, wherein the internal threads of the slider are coated with a friction-reducing coating.

6. The implantable annuloplasty device of claim 2, wherein at least one of the sliders is electropolished before application of a friction-reducing coating thereto.

7. The implantable annuloplasty device of claim 6, wherein a slider screw associated with the at least one slider is anodized.

8. The implantable annuloplasty device of claim 7, wherein the slider screw associated with the at least one slider is coated with a friction-reducing coating.

9. The implantable annuloplasty device of claim 6, wherein the slider screw associated with the at least one slider is coated with a friction-reducing coating.

10. The implantable annuloplasty device of claim 1, wherein:
the plurality of movable components include at least one anchor movable with respect to the frame member to secure the frame member to an implant site, and an associated anchor housing mounted to the frame member and through which the at least one anchor extends; and
at least a portion of a surface of the anchor housing is coated with a friction-reducing coating.

11. The implantable annuloplasty device of claim 10, wherein the anchor housing is smoothened before application of the friction-reducing coating thereto.

12. The implantable annuloplasty device of claim 10, wherein the anchor housing is tumbled before application of the friction-reducing coating thereto.

13. The implantable annuloplasty device of claim 1, wherein the coated component is formed of a metal and the friction-reducing coating is a polytetrafluoroethylene coating.

14. The implantable annuloplasty device of claim 1, wherein the coated surface is an untreated surface which has not been subjected to a pre-treatment process recommended for acceptance of the friction-reducing coating on the surface prior to application of the friction-reducing coating thereto.

15. An implantable device and associated deployment system, the device and system comprising:
an implantable device comprising at least one component movable with respect to another component of the implantable device; and
a deployment system configured to adjust the position of the at least one movable component;
wherein at least a portion of at least one of the movable component or a component adjacent the movable component is coated with a friction-reducing coating without having been subjected to a pre-treatment process recommended for acceptance of the friction-reducing coating on the surface prior to application of the friction-reducing coating thereto.

16. The device and system of claim 15, wherein the coated surface is smoothened before application of a friction-reducing coating thereto.

17. The device and system of claim 15, wherein:
the implantable device comprises a frame member and at least one of a component movable with respect to the frame member to secure the frame member to tissue, or a component movable with respect to the frame member to adjust a configuration of the frame member; and
the deployment system comprises at least one latch actuator engageable with an associated latch on the at least one movable component, the latch on the movable components not being coated with a friction-reducing coating.

18. A method of reducing friction between components of an implantable device, the method comprising applying a friction-reducing coating to at least a portion of at least one component of the implantable device which is movable with respect to another component of the implantable device without pre-treating the portion to which the friction-reducing coating is applied to facilitate acceptance of the friction-reducing coating on the portion.

19. The method of claim 18, further comprising allowing the efficacy of the friction-reducing coating to be reduced as the movable component is moved.

20. The method of claim 18, further comprising smoothening the surface of the at least one portion of the at least one component prior to applying the friction-reducing coating thereto.
